(19) United States
(12) Patent Application Publication
(10) Pub. No.: US 2007/0202071 A1
(43) Pub. Date: Aug. 30, 2007

(54) Emulsions Comprising A Dendritic Polymer And Use Of A Dendritic Polymer As An Emulsification Agent

(76) Inventors: Mikel Morvan, Princeton (FR); Alain Senechal, Charenton (FR); Olivier Anthony, Meriel (FR); Franck Touraud, Vernon (FR); Jean-Francois Sassi, Saint-Romain En Jarez (FR); Katerina Karagianni, Paris (FR); Rene Rossin, Oullins (FR)

Correspondence Address: Jean Louis Seugnet;Rhodia Inc, 8 Cedar Brook Drive, Cranbury, NJ, 08512-7500, US

(21) Appl. No.: 10/573,846

(22) PCT Filed: Sep. 29, 2004
(86) PCT No.: PCT/FR04/02461

§ 371 Date: Oct. 31, 2006

Related U.S. Application Data
(60) Provisional application No. 60/506,788, filed on Sep. 29, 2003.

(51) Int. Cl. A61K 8/87; A61K 8/73; C08F 20/00

(52) U.S. Cl. 424/70.17; 525/440; 977/754; 525/438; 525/437

(57) ABSTRACT

The invention relates to novel emulsions comprising a dendritic polymer. The invention also relates to the use of a dendritic polymer as an emulsification agent. The inventive emulsions are suitable for use in the cosmetics, detergents, paint and coatings industries.

The subject of the present invention is novel emulsions comprising a dendritic polymer. Its subject is also the use of a dendritic polymer as emulsifying agent.

Emulsions are physicochemical structures or systems which find application in numerous fields. Reference is also made to formulations in the form of emulsions. The fields of application include in particular cosmetic formulations, detergent formulations, formulations for coatings (paint and the like), certain methods of polymerization (preparation of latex, preparation of polymers or copolymers based on polyacrylamide), plant-protection formulations. Emulsions are also a means of vectorizing or protecting a compound (inner phase or compound contained in the inner phase).

An emulsion comprises at least two immiscible liquid phases, one outer phase and one inner phase dispersed in the form of droplets in the outer phase. Often, one of the two phases is an aqueous phase. If the outer phase is aqueous, the emulsion is often said to be a direct emulsion or an “oil-in-water” emulsion. If the inner phase is aqueous, the emulsion is often said to be an inverse emulsion or a “water-in-oil” emulsion. An emulsion also generally comprises an emulsifying agent which plays a role at the interfaces of the droplets. An emulsion is generally prepared by mixing more or less vigorously two phases and, where appropriate, the emulsifying agent. If the mixture obtained is at thermodynamic equilibrium, the emulsion is generally said to be a microemulsion. If the mixture obtained is not at thermodynamic equilibrium, energy having been given to the system by mixing, the emulsion is generally simply said to be an emulsion. In the present application, the term “emulsion” of course covers emulsions and also microemulsions.

The size of the droplets and their stability over time depend in particular on the nature and the quantity of the various phases and of the emulsifying agent. They also generally depend on the strength of the mixing performed for their production (quantity of energy given to the system). Thus, an emulsifying agent and its quantity may be chosen according to the phases to be emulsified.

Numerous emulsifying agents are known. Among the most widely used, there may be mentioned surfactants. They are often molecules of relatively low molecular weight, comprising a hydrophilic part and a hydrophobic part. These agents can have disadvantages in some applications. They are often irritant, which has a major disadvantage for example in the cosmetic and pharmaceutical fields. They can furthermore have a negative impact on the environment. Their presence in some formulations, in particular in coating formulations like paints, can induce migration phenomena at the interfaces and thus induce problems of appearance and color with the coated object. Finally, they are often highly foaming, which is not always desired for the formulation and can lead to difficulties during the preparation of a formulation.

Polymeric emulsifying agents are also known. There may be mentioned for example poly(ethylene oxide)/poly(propylene oxide)/poly(ethylene oxide) block copolymers used for the production of direct emulsions. There may also be mentioned copolymers of the polyhydroxystearate/PEG/polyhydroxystearate type, for example marketed under the name Arlacel or Superonic, by Uniquema, used for the production of inverse emulsions. It is also known to use polysaccharides and polysaccharide derivatives. These polymeric agents provide solutions for emulsifying specific systems for which there is no sufficiently effective surfactant (quantity introduced, stability over time and the like), or for which a surfactant would have disadvantages, such as those which were mentioned above. However, the possibilities for using these polymeric compounds are limited. For example, they may exhibit low resistance at high temperature, or high degradability in formulations containing enzymes.

The subject of the present invention is novel emulsions, novel in particular by virtue of the emulsifying agent, constituting an alternative to known emulsions. Its subject is thus the novel use of a polymeric compound as emulsifying agent. The emulsions according to the invention, and the use according to the invention, have in particular the advantage of low foaming, and/or resistance at high temperature, and/or low degradability in formulations containing enzymes and/or high versatility of use. The emulsions according to the invention, and the use according to the invention, have the advantage, for inverse emulsions, of allowing the production of stable, small-sized dispersions. Furthermore, the emulsions according to the invention have the advantage of being stable in a wide variety of media. They are additionally stable when the outer phase is an aqueous phase, which may contain a wide variety of products. They can be used in the presence of a significant quantity of a detergent such as a surfactant, for example an anionic surfactant. Under certain conditions and in some formulations, the emulsifying agent may be adsorbed on surfaces, and may thus serve as a vector for depositing the inner phase on a surface. In particular., such a vectorization by the emulsifying agent is not shielded by the presence of anionic surfactants. This is particularly useful for laundry soaps or shampoos.

Thus, the invention provides an emulsion comprising an inner phase, an outer phase and an emulsifying polymer, one of the phases being an aqueous phase, wherein the emulsifying polymer is a dendritic polymer.

Likewise, the invention proposes the use of a dendritic polymer as emulsifying agent.

It is specified that the emulsions according to the invention comprise the dendritic polymer as emulsifying agent, but that it is not impossible for them to further comprise one or more other emulsifying agents. Reference is sometimes made to coemulsifiers or emulsifier booster, for example surfactant booster. In the context of inverse emulsions, the dendritic polymer is advantageously used as sole emulsifying agent.

Phases of the Emulsion

The emulsion comprises at least two immiscible liquid phases, an inner phase and an outer phase, one of which is aqueous. It is not impossible for the emulsion to comprise three immiscible phases, the emulsions then having an aqueous phase, a first group of droplets (first inner phase) dispersed in the outer phase, and a second group of droplets (second inner phase) dispersed in the outer phase. It is not impossible either for a phase (aqueous or nonaqueous phase) that is immiscible with the inner phase to be dispersed in the form of droplets within the droplets of the inner phase. In this case, reference is often made to multiple emulsions, comprising an inner emulsion and an outer emulsion. For example, this may be water-in-oil-in-water emulsions comprising an inner phase (water), an intermediate phase (oil) and an outer phase. The dispersion of the inner phase in the intermediate phase constitutes an inner inverse emulsion, the dispersion of the intermediate phase in the outer phase constitutes an outer direct emulsion. Likewise, in the present application, reference may be made to inner or outer emulsifying agent. In the present application, the notion of inner emulsion covers both a simple inverse emulsion and an inner inverse emulsion of a multiple emulsion. The notion of direct emulsion covers both a simple direct emulsion and an outer direct emulsion of a multiple emulsion.

Aqueous Phase

The aqueous phase may be an outer phase, where appropriate an outer phase of a multiple emulsion. Reference is made to direct emulsions. The aqueous phase may be an inner phase, where appropriate the outer phase of a multiple emulsion. Reference is made to inverse emulsions. The aqueous phase of course comprises water, and where appropriate other compounds. The other compounds may be solvents or cosolvents, dissolved or solid compounds dispersed in water, for example active substances. The expression “other compounds” of the aqueous phase does not refer to the liquid inner phase or to the intermediate phase of a multiple emulsion.

The dendritic polymer is preferably dispersible or soluble in water.

The aqueous phase may additionally contain compounds intended to confer a certain pH on the solution, and/or salts which substantially have no influence on the pH. It is specified that the pH may have an influence on the water-solubility of the dendritic polymer and on the hydrophilicity of groups contained in the dendritic polymer. This is the case in particular for the carboxylic acid groups, and for the amine groups. It is preferable to adopt pH and concentration conditions such that the dendritic polymer is water-dispersible or water-soluble, and/or such that groups sensitive to pH are in ionic form. While there is a pH limit above or below which the dendritic polymer is dispersible or soluble, the pH is preferably in the range from the limit to 2 units above or below the limit, in the dispersibility or solubility range. Such conditions and such groups are detailed below, in relation to the description of the dendritic polymers.

The aqueous phase may also comprise compounds customarily used in the fields of formulations in the form of emulsions or comprising emulsions, for example in the fields of domestic care (detergents, laundry soaps, cleaning of hard surfaces, dishes), in the cosmetic fields (hair care; shampoo; shower gels; creams; milks; lotions; gels; deodorants), in the industrial fields (emulsion polymerization, treatment of surfaces in industrial processes, lubrication and the like), in the fields of coatings, for example in paints. These may also be anionic, cationic, amphoteric, zwitterionic or nonionic surfactants, builders, hydrophilic active agents, salts and viscosity-promoting agents.

Nonaqueous Phase

The emulsion comprises a phase that is immiscible with the aqueous phase. For the sake of simplicity, this phase will be designated “nonaqueous phase” or “oil phase”, or “hydrophobic phase”. The expression immiscible phases is understood to mean that a phase is not soluble at more than 10% in the other phase, at a temperature of 20° C. The nonaqueous phase may be the inner phase (direct emulsions), or the outer phase (inverse emulsions). This may be in particular an intermediate phase of a multiple emulsion.

Examples of compounds constituting the nonaqueous phase, or contained in the nonaqueous phase include:
organic oils/fat/waxes of animal origin or of plant origin;
mineral oils/waxes, for example hydrocarbon-based paraffins;
products derived from the alcoholysis of the abovementioned oils and optionally from a subsequent esterification;
the products derived from the transesterification of the abovementioned oils;
essential oils;
mono-, di- and triglycerides;
saturated or unsaturated fatty acids comprising 10 to 40 carbon atoms; esters of such acids and of an alcohol comprising 1 to 6 carbon atoms;
saturated or unsaturated monoalcohols comprising 2 to 40 carbon atoms;
polyols comprising 2 to 10 carbon atoms;
silicones, in particular aminosilicones;
hydrocarbons or hydrocarbon cuts;
monomers that are insoluble in water, in particular used for the polymerizations of isocyanate with polyols or for the polymerizations of latex,
precursors of resins or macromolecules insoluble in water, such as alkyd or isocyanate compounds.

As organic oils/fat/waxes of animal origin, there may be mentioned, inter alia, sperm whale oil, whale oil, seal oil, shark oil, cod-liver oil, lard, mutton fat (tallow), perhdyrosqualene, beeswax, alone or as a mixture.

By way of examples of organic oils/fat/waxes of plant origin, there may be mentioned, inter alia, rapeseed oil, sunflower oil, peanut oil, olive oil, nut oil, corn oil, soybean oil, avocado oil, linseed oil, hemp oil, grapeseed oil, copra oil, palm oil, cottonseed oil, babassu oil, jojoba oil, sesame oil, castor oil, macadamia oil, sweet almond oil, carnauba wax, shea butter, cocoa butter, peanut butter, alone or as a mixture.

As regards the mineral oils/waxes, there may be mentioned, inter alia, naphthenic oil, paraffin oil (petroleum jelly), isoparaffin oil, paraffin waxes, alone or as a mixture.

The products derived from alcoholysis of the abovementioned oils may also be used.

Among the essential oils, there may be mentioned, with no limitation being implied, the oils and/or essences of mint, spearmint, peppermint, menthol, vanilla, cinnamon, bay, aniseed, eucalyptus, thyme, sage, cedar leaf, nutmeg, citrus (lemon, lime, grapefruit, orange), fruits (apple, pear, peach, cherry, plum, strawberry, raspberry, apricot, pineapple, grape and the like), alone or as mixtures.

As regards the fatty acids, the latter, which are saturated or unsaturated, comprise 10 to 40 carbon atoms, more particularly 18 to 40 carbon atoms, and may comprise one or more ethylenic unsaturations, conjugated or unconjugated. It should be noted that said acids may comprise one or more hydroxyl groups.

As examples of saturated fatty acids, there may be mentioned palmitic, stearic and behenic acids.

As examples of unsaturated fatty acids, there may be mentioned myristoleic, palmitoleic, oleic, erucic, linoleic, linolenic, arachidonic and ricinoleic acids, and mixtures thereof.

As regards the fatty acid esters, there may be mentioned the esters of the acids listed above, for which the portion derived from the alcohol comprises 1 to 6 carbon atoms, such as methyl, ethyl, propyl and isopropyl esters, and the like. As an example of alcohols of these esters, there may be mentioned ethanol and those corresponding to the abovementioned acids. Among the suitable polyols for these esters, glycerol may be preferably mentioned.

The nonaqueous phase may comprise a silicone or a mixture of several of them. Reference is often made to silicone oils. The aminosilicones are in particular useful in the fields of detergents. Further details are given below regarding the silicones.

They may be in particular an oil, a wax or a resin as a linear, cyclic, branched or crosslinked polyorganosiloxane.

Said polyorganosiloxane preferably has a dynamic viscosity, measured at 25° C. and at the shear rate of 0.01 Hz for a stress of 1500 Pa (performed on a Carrimed® of type CSL2-500), of between 10⁴ and 10⁹ cP.

It may be in particular:
a nonionic polyorganosiloxane
a polyorganosiloxane having at least one cationic or potentially cationic functional group
a polyorganosiloxane having at least one anionic or potentially anionic functional group
an amphoteric polyorganosiloxane having at least one cationic or potentially cationic functional group and at least one anionic or potentially anionic functional group.

Preferably, it is a nonionic or amino polyorganosiloxane.

By way of examples of polyorganosiloxanes, there may be mentioned:
linear, cyclic or crosslinked polyorganosiloxanes formed of nonionic organosiloxane units of general formula
   (R)ₐ(X)_{b}Si(O)_{[4-(a+b)]/2} (I)
   in which formula
the symbols R are identical or different and represent a linear or branched alkyl hydrocarbon radical having from 1 to 4 carbon atoms, an aryl, in particular phenyl, radical;
the symbols X are identical or different and represent a hydroxyl group, a linear or branched alkoxy radical having from 1 to 12 carbon atoms, a functional group OCOR′, where R′ represents an alkyl groups containing from 1 to 12 carbon atoms, preferably 1 carbon atom;
a is equal to 0, 1, 2 or 3
b is equal to 0, 1, 2 or 3
a+b is equal to 0, 1, 2 or 3.

Preferably, said polyorganosiloxane is at least substantially linear, and most preferably linear. By way of example, there may be mentioned in particular the oils α,ω-bis(hydroxy)polydimethylsiloxanes, the oils α,ω-bis(trimethyl)polydimethylsiloxanes, cyclic polydimethylsiloxanes, polymethylphenylsiloxanes,
the linear, cyclic or crosslinked polyorganosiloxanes. comprising, per mole, at least one ionic or nonionic organosiloxane unit of general formula
   (R)ₐ(X)_{b}(B)_{c}Si(O)_{[4-(a+b+c)]/2} (II)
   in which formula
the symbols R are identical or different and represent a linear or branched monovalent alkyl hydrocarbon radical having from 1 to 4 carbon atoms, an aryl, in particular phenyl, radical;
the symbols X are identical or different and represent a hydroxyl group, a linear or branched alkoxy radical having from 1 to 12 carbon atoms, a functional group OCOR′, where R′ represents an alkyl group containing from 1 to 12 carbon atoms, preferably 1 carbon atom;
the symbols B are identical or different and represent an aliphatic and/or aromatic and/or cyclic hydrocarbon radical containing up to 30 carbon atoms, which is optionally interrupted by one or more oxygen and/or nitrogen and/or sulfur heteroatoms, which optionally carries one or more ether, ester, thiol, hydroxyl, optionally quaternized amine, and carboxylate functional groups, the symbol B being attached to the silicon preferably by means of an Si—C— bond;
a is equal to 0, 1 or 2
b is equal to 0, 1 or 2
c is equal to 1 or 2
a+b+c is equal to 1, 2 or 3.

By way of example of substituents corresponding to the symbol (B) in formula (II) above, there may be mentioned
the polyether groups of formula
   —(CH₂)ₙ—(OC₂H₄)ₘ—(OCH₃H₆)ₚ—OR′
   where n is equal to 2 or 3, m and p each range from 0 to 30 and R′ represents an alkyl residue containing from 1 to 12 carbon atoms, preferably 1 to 4 carbon atoms,
the primary, secondary, tertiary or quaternized amino groups such as those of formula
   R¹—N(R²)(R³)
   where
the symbol R¹ represents an alkylene group containing from 2 to 6 carbon atoms, which is optionally substituted or interrupted by one or more nitrogen or oxygen atoms,
the symbols R² and R³, which are identical or different, represent
   H,
   an alkyl or hydroxyalkyl group containing from 1 to 12 carbon atoms, preferably from 1 to 6 carbon atoms,
   an aminoalkyl, preferably a primary aminoalkyl, group in which the alkyl group contains from 1 to 12 carbon atoms, preferably from 1 to 6 carbon atoms, which is optionally substituted and/or interrupted by at least one nitrogen and/or oxygen atom,
      said amino group being optionally quaternized, for example, with a hydrohalic acid or an alkyl or aryl halide.

There may be mentioned in particular those of formulae
—(CH₂)₃NH₂
—(CH₂)₃NH₃⁺X⁻
—(CH₂)₃N(CH₃)₂
—(CH₂)₃N⁺(CH₃)₂(C₁₆H₃₇)X⁻
—(CH₂)₃NHCH₂CH₂NH₂
—(CH₂)₃N(CH₂CH₂OH)₂
—(CH₂)₃N(CH₂CH₂NH₂)₂

Preferably, the polyorganosiloxanes carrying amino functional groups have in their chain, per 100 silicon atoms in total, from 0.1 to 50, preferably from 0.3 to 10, most particularly from 0.5 to 5 amino functionalized silicon atoms,
the sterically hindered piperidinyl groups of formula III 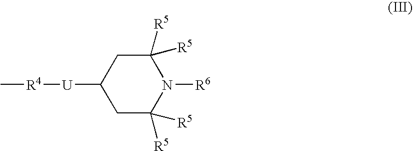
where
   R⁴ is a divalent hydrocarbon radical chosen from:
      linear or branched alkylene radicals having 2 to 18 carbon atoms;
      alkylenecarbonyl radicals in which the linear or branched alkylene part contains 2 to 20 carbon atoms;
      alkylenecyclohexylene radicals in which the linear or branched alkylene part contains 2 to 12 carbon atoms and the cyclohexylene part contains an OH group and optionally 1 or 2 alkyl radicals having 1 to 4 carbon atoms;
      the radicals of formula —R⁷—O—R⁷ in which the radicals R⁷, which are identical or different, represent alkylene radicals having 1 to 12 carbon atoms;
      the radicals of formula —R⁷—O—R⁷ in which the radicals R⁷ have the meanings indicated above and one if them or both are substituted with one or two —OH group(s);
      the radicals of formula —R⁷—COO—R⁷ in which the radicals R⁷ have the meanings indicated above;
      the radicals of formula —R⁸—O—R⁹—O—CO—R⁸ in which the radicals R⁸ and R⁹, which are identical or different, represent alkylene radicals having 2 to 12 carbon atoms and the radical R⁹ is optionally substituted with a hydroxyl radical;
      U represents —O— or —NR¹⁰—, R¹⁰ being a radical chosen from a hydrogen atom, a linear or branched alkyl radical containing 1 to 6 carbon atoms and a divalent radical of formula: 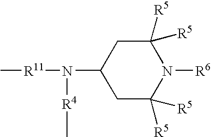
      in which R⁴ has the meaning indicated above, R⁵ and R⁶ have the meanings indicated below and R¹¹ represents a linear or branched divalent alkylene radical having from 1 to 12 carbon atoms, one of the valency bonds (that of R¹¹) being linked to the atom of —NR¹⁰—, the other (that of R⁴) being linked to a silicon atom;
   the radicals R⁵ are identical or different, chosen from linear or branched alkyl radicals having 1 to 3 carbon atoms and the phenyl radical;
   the radical R⁶ represents a hydrogen radical or the radical R⁵ or O═;
or the sterically hindered piperidinyl groups of formula IV 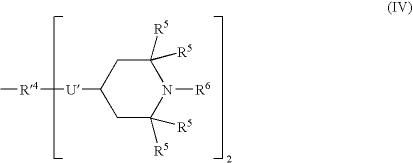
   R′⁴ is chosen from a trivalent radical of formula: 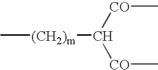
   where m represents a number from 2 to 20, and a trivalent radical of formula: 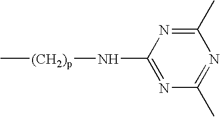
   where p represents a number from 2 to 20;
      U′ represents —O— or NR¹², R¹² being a radical chosen from a hydrogen atom, a linear or branched alkyl radical containing 1 to 6 carbon atoms;
R⁵ and R⁶ have the same meanings as those given above in relation to formula III.

Preferably, said polyorganosiloxane with a sterically hindered amino functional group is a linear, cyclic or three-dimensional polyorganosiloxane of formula (V): 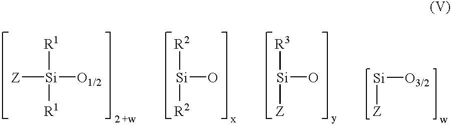
in which:
(1) the symbols Z, which are identical or different, represent R¹ above and/or the symbol B below;
(2) the symbols R¹, R² and R³, which are identical and/or different, represent a monovalent hydrocarbon radical chosen from linear or branched alkyl radicals having from 1 to 4 carbon atoms, linear or branched alkoxy radicals having from 1 to 4 carbon atoms, a phenyl radical and, preferably, a hydroxyl radical, an ethoxy radical, a methoxy radical or a methyl radical;
(3) the symbols B, functional groups which are identical and/or different, represent a group with sterically hindered piperidinyl functional group(s) which is chosen from those mentioned above; and
(4)—the number of organosiloxy units with no group B ranges from 10 to 450, preferably from 50 to 250;
   the number of organosiloxy units with a group B ranges from 1 to 5, preferably from 1 to 3;
   0≦w≦10 and 8<x<448.

Most preferably, said polyorganosiloxane is linear.

By way of example of commercially available polyorganosiloxane products which may be used as hydrophobic phase (A), there may be mentioned in particular the oils RHODORSIL® 21645, RHODORSIL® Extrasoft marketed by Rhodia.

The nonaqueous phase may comprise monomers which are insoluble in water, which can be used in particular for emulsion polymerization processes, for example for the manufacture of latex.

Finally, it is specified that it is not impossible for the nonaqueous phase to contain a quantity of water, or of water-soluble monomers, which does not exceed the limit of solubility of water or of monomers in said phase.

Examples of monomers which may constitute the nonaqueous phase, or which may be contained in said phase, include, alone or as mixtures:
esters of linear or branched, cyclic or aromatic mono- or polycarboxylic acids comprising at least one ethylenic unsaturation;
esters of saturated carboxylic acids comprising 8 to 30 carbon atoms, optionally carrying a hydroxyl group;
α,β-ethylenically unsaturated nitrites, vinyl ethers, vinyl esters, vinylaromatic monomers, vinyl or vinylidene halides;
aromatic or nonaromatic, linear or branched hydrocarbon monomers comprising at least one ethylenic unsaturation;
macromonomers derived from such monomers.

There may be mentioned more particularly:
esters of (meth)acrylic acid with an alcohol comprising 1 to 12 carbon atoms such as methyl (meth)acrylate, ethyl (meth)acrylate, propyl (meth)acrylate, n-butyl (meth)acrylate, t-butyl (meth)acrylate, isobutyl (meth)acrylate, 2-ethylhexyl acrylate, isodecyl acrylate;
vinyl acetate, Vinyl versatate®, vinyl propionate, vinyl chloride, vinylidene chloride, methyl vinyl ether, ethyl vinyl ether;
the vinyl nitriles include more particularly those having from 3 to 12 carbon atoms, such as in particular acrylonitrile and methacrylonitrile;
styrene, α-methylstyrene, vinyltoluene, butadiene, chloroprene.

It should be noted that the nonaqueous inner phase may comprise an aqueous or nonaqueous phase dispersed in the form of an emulsion inside it. The emulsion is then a multiple emulsion.

Dendritic Polymer

The emulsion according to the invention comprises a dendritic polymer as emulsifying agent. The term “dendritic polymer” refers to macromolecular compounds comprising several branches. This may be regular dendrimers or hyperbranched polymers.

The dendritic polymer comprises hydrophobic groups and hydrophilic groups. The hydrophobic groups may be contained in repeating units inside the polymer. These may be, for example, at least divalent alkylene groups with at least 3 consecutive carbon atoms, or at least divalent groups comprising a phenyl unit, for example the phenylene group. This is advantageously a group of formula —(CH₂)ₙ— where n is greater than or equal to 3, for example 4, 5, 6 or 11, and/or a group of formula —C₆H₄—.

The hydrophilic groups may be contained in repeating units inside the polymer and/or may be included at the end of the polymer chains. When the emulsion is a direct emulsion, the aqueous phase being the outer phase, at least part of the hydrophilic, or potentially hydrophilic, groups are advantageously groups present at the end of polymer chains. The hydrophilic groups contained in repeating units are often considered as functional groups for polymerizations. They are for example groups, or functional groups, of formulae —COO-(polyesters), —O-(polyethers), —CONH-(polyamide), —OCOO-(polycarbonate), —NH—COO-(polyurethane), —N<(polyamine), —NH—CO—NH-(urea), —CO—NH—CO-(imide).

It should be noted that it is not impossible for the ends of polymer chains to comprise hydrophobic groups, such as alkyl groups. The presence of such groups can help to modulate the emulsifying properties of the dendritic polymer.

The hydrophobic groups may be contained in repeating units inside the polymer and/or may be included at the end of the polymer chains. When the emulsion is an inverse emulsion, the aqueous phase being the inner phase, at least part of the hydrophobic groups are advantageously groups present at the ends of polymer chains. It should be noted that it is not impossible for the ends of polymer chains to include hydrophilic, or potentially hydrophilic, groups. The presence of such groups may help to modulate the emulsifying properties of the dendritic polymer.

When the emulsion is a multiple emulsion comprising an inner aqueous phase, an intermediate phase and an outer aqueous phase, the inner phase and the intermediate phase constituting an inner inverse emulsion, the intermediate phase and the outer phase constituting an outer direct emulsion, and when the outer direct emulsion and the inner inverse emulsion comprise the dendritic polymer, the latter preferably comprises hydrophobic groups and hydrophilic (or potentially hydrophilic) groups at the end of the polymer chains.

The dendritic polymer may preferably comprise hydrophilic or potentially hydrophilic groups (depending for example on the pH) at the ends of the polymer chains. Furthermore, the nature and the properties of these groups may be more easily controlled, modified or varied either during the polymerization or later, by post-functionalization.

The dendritic polymer may preferably comprise hydrophobic groups at the ends of the polymer chains. Furthermore, the nature and the properties of these groups may be more easily controlled, modified or varied, either during the polymerization or later, by post-functionalization.

Examples of hydrophilic groups include:
acid groups such as sulfonic groups, phosphonic groups, carboxylic acid groups and their basic sulfonate, phosphate, phosphonate and carboxylate forms,
primary, secondary and tertiary amine groups, their acidic ammonium forms, and quaternary ammonium groups.

It should be mentioned that the hydrophilicity of a group may depend on the pH. In the present application, the expression hydrophilic group denotes groups which are hydrophilic at any pH, and groups whose hydrophilicity depends on the pH (potentially hydrophilic groups).

Examples of hydrophobic groups include:
saturated or unsaturated alkyl groups,
aryl, aralkyl or alkylaryl groups, for example phenyl or naphthyl,
silicone or silane groups,
fluorinated groups.

Examples of dendritic polymers include:
dendrimers with a polypropyleneimine backbone, such as the Straburst® range marketed by the company DSM,
dendrimers with a polyamidoester (or polyester amide) backbone, such as the Hybrane® range marketed by the company DSM,
dendrimers with a polyamidoamine (PAMAM) backbone,
polyether dendrimers,
hyperbranched diaminobutane-aminopropyl DAB(PA)ₙ polymers,
hyperbranched polyesters such as the BOLTORN® range marketed by the company Perstorp.

The hyperbranched polyesters and the hyperbranched polyamides are in particular dendritic polymers which are particularly suitable for carrying out the invention.

According to an advantageous embodiment, the dendritic polymer is a polymer capable of being obtained by a process comprising the following steps:

Step a) polycondensation of at least one plurifunctional monomer of formula (I) comprising at least three reactive polycondensation functional groups,
A-R—(B)ᵣ (I)
in which formula
f is an integer greater than or equal to 2, preferably ranging from 2 to 10, most particularly equal to 2,
the symbol A represents a reactive functional group or a group carrying a reactive functional group chosen from amino, carboxyl, hydroxyl, oxiranyl, halo and isocyanato functional groups, or precursors thereof,
the symbol B represents a reaction functional group or a group carrying a reactive functional group chosen from amino, carboxyl, hydroxyl, oxiranyl, halo and isocyanato functional groups, or precursors thereof, which is an antagonist of A,
the symbol R represents a linear or branched aliphatic, cycloaliphatic or aromatic polyvalent hydrocarbon residue containing from 1 to 50, preferably from 3 to 20 carbon atoms, optionally interrupted by one or more oxygen, nitrogen, sulfur or phosphorus heteroatoms, said residue optionally carrying functional groups not capable of reacting with the functional groups A and B,
   Step b) optionally at least partial hydrophilic functionalization of the polymer obtained in the polycondensation step.

The symbol B represents a reactive functional group which is an antagonist of the reactive functional group A; this means that the functional group B is capable of reacting with the functional group A by condensation.

Thus, the functional groups which are antagonists
of an amino functional group, are in particular the functional groups carboxyl (formation of an amide), isocyanato (formation of a urea), oxiranyl (formation of a secondary or tertiary β-hydroxylated amine),
of a carboxyl functional group, are in particular the functional groups amino (formation of an amide), hydroxyl (formation of an ester), isocyanato (formation of an amide),
of a hydroxyl functional group, are in particular the functional groups carboxyl (formation of an ester), oxiranyl (formation of an ether), isocyanato (formation of an amide),
of an oxiranyl functional group, are in particular the functional groups hydroxyl (formation of an ether), carboxyl (formation of an ester), amino (formation of a secondary or tertiary β-hydroxylated amine),
of an isocyanato functional group, are in particular the amino, hydroxyl and carboxyl functional groups,
of a halo functional. group, are in particular the hydroxyl functional groups.

Among the precursors of an amino functional group, amine salts, such as hydrochlorides, may be mentioned.

Among the precursors of a carboxyl functional group, there may be mentioned in particular esters, preferably as C1-C4, most particularly C1-C2, acid halides, anhydrides, amides.

Among the precursors of a hydroxyl functional group, epoxy groups may be mentioned in particular.

According to a variant embodiment, said polycondensation operation is additionally performed in the presence:
of at least one bifunctional monomer, in linear form, of formula (II) or in the corresponding cyclic form, comprising two polycondensation/polymerization reactive functional groups
   A′-R′—B′ (II)
in which formula:
   the symbol A′, which is identical to or different from A, represents a reactive functional group chosen from amino, carboxyl, hydroxyl, oxiranyl, halo and isocyanato functional groups, or precursors thereof, which is an antagonist of B and B′,
   the symbol B′, which is identical to or different from B, represents a reactive functional group chosen from amino, carboxyl, hydroxyl, oxiranyl, halo and isocyanato functional groups, or precursors thereof, which is an antagonist of A and A′,
   the symbol R′, which is identical to or different from R, represents a linear or branched aliphatic, cycloaliphatic or aromatic polyvalent hydrocarbon residue containing from 1 to 50, preferably from 3 to 20 carbon atoms, optionally interrupted by one or more oxygen, nitrogen, sulfur or phosphorus heteroatoms, said residue optionally carrying functional groups not capable of reacting with the functional groups A, A′, B and B′,
   the reactive functional group A′ being capable of reacting with the functional group B and/or the functional group B′ by condensation;
   the reactive functional group B′ being capable of reacting with the functional group A and/or the functional group A′ by condensation;
and/or of at least one “core” monomer of formula (III), comprising at least one functional group capable of reacting, by condensation, with the monomer of formula (I) and/or the monomer of formula (II)
   R¹—(B″)ₙ (III)
in which formula
   n is an integer greater than or equal to 1, preferably ranging from 1 to 100, most particularly from 1 to 20,
   the symbol B″ represents a reactive functional group, which is identical to or different from B or B′, chosen from amino, carboxyl, hydroxyl, oxiranyl, halo and isocyanato functional groups, or precursors thereof, which is an antagonist of A and A′,
   the symbol R¹ represents a linear or branched aliphatic, cycloaliphatic or aromatic polyvalent hydrocarbon residue containing from 1 to 50, preferably from 3 to 20 carbon atoms, optionally interrupted by one or more oxygen, nitrogen, sulfur or phosphorus heteroatoms, or an organosiloxane or polyorganosiloxane residue, said residue R¹ optionally carrying functional groups not capable of reacting with the functional groups A, A′, B, B′ and B″,
   the reactive functional group B″ being capable of reacting with the functional group A and/or the functional group A′ by condensation;
and/or of at least one “chain limiting” mono-functional monomer of formula (IV)
   A″-R² (IV)
in which formula
   the symbol A″ represents a reactive functional group, which is identical to or different from A or A′, chosen from amino, carboxyl, hydroxyl, oxiranyl, halo and isocyanato functional groups, or precursors thereof, which is an antagonist of B, B′ and B″,
   the symbol R² represents a linear or branched aliphatic, cycloaliphatic or aromatic polyvalent hydrocarbon residue containing from 1 to 50, preferably from 3 to 20 carbon atoms, optionally interrupted by one or more oxygen, nitrogen, sulfur or phosphorus heteroatoms, or an organosiloxane or polyorganosiloxane residue, said residue R² optionally carrying functional groups not capable of reacting with the functional groups A, A′, A″, B, B′ and B″,
   the reactive functional group A″ being capable of reacting with the functional group B and/or the functional group B′ and/or the functional group B″ by condensation;
at least one of the reactive functional groups of at least one of the monomers of formula (II), (III) or (IV) being capable of reacting with a functional group which is an antagonist of the plurifunctional monomer of formula (I).

Preferably, the functional groups A, A′, A″ and B, B′, B″ are chosen from reactive functional groups or a group carrying reactive functional. groups chosen from amino, carboxyl, hydroxyl and oxiranyl functional groups, or precursors thereof. More preferably still, said functional groups are chosen from reactive functional groups or a group carrying reactive amino and carboxyl functional groups, or precursors thereof.

For proper implementation of the invention:
the molar ratio of the monomer of formula (I) to the monomer of formula (II) is advantageously greater than 0.05, preferably ranges from 0.125 to 2;
the molar ratio of the monomer of formula (III) to the monomer of formula (I) is advantageously less than or equal to 1, preferably less than or equal to 1/2, and more preferably still ranges from 0 to 1/3; said ratio ranges most particularly from 0 to 1/5;
the molar ratio of the monomer of formula (IV) to the monomer of formula (I) is advantageously less than or equal to 10, preferably less than or equal to 5; said ratio ranges most particularly from 0 to 2, when f is equal to 2.

The elementary entity considered for defining the various molar ratios is the molecule.

It goes without saying that the expression “condensation reaction” also includes the notion of addition reaction when one or more functional groups which are antagonists of at least one of the monomers used is contained in a ring (lactams, lactones, epoxides for example).

By way of example of monomer (I), there may be mentioned:
5-aminoisophthalic acid
6-aminoundecanedioic acid,
3-aminopimelic diacid,
aspartic acid,
glutamic acid,
3,5-diaminobenzoic acid,
3,4-diaminobenzoic acid,
lysine,
α,α-bis(hydroxymethyl)propionic acid,
α,α-bis(hydroxymethyl)butyric acid,
α,α,α-tris(hydroxymethyl)acetic acid,
α,α-bis(hydroxymethyl)valeric acid,
α,α-bis(hydroxy)propionic acid,
3,5-dihydroxybenzoic acid,
or mixtures thereof.

By way of example of bifunctional monomer of formula (II), there may be mentioned:
ε-caprolactam,
aminocaproic acid,
para- or meta-aminobenzoic acid,
11-aminoundecanoic acid,
lauryllactam,
12-aminododecanoic acid,
hydroxyacetic acid (glycolic acid),
hydroxyvaleric acid,
hydroxypropionic acid,
hydroxypivalic acid,
glycolide,
δ-valerolactone,
β-propiolactone,
ε-caprolactone,
lactide,
lactic acid,
or mixtures thereof.

More preferably, the bifunctional monomers of formula (II) are the monomers used for the manufacture of linear thermoplastic polyamides. Thus, there may be mentioned ω-aminoalkanoic compounds containing a hydrocarbon chain having from 4 to 12 carbon atoms, or lactams derived from these amino acids such as ε-caprolactam. The bifunctional monomer preferred for carrying out the invention is ε-caprolactam.

According to an advantageous modality of the invention, at least some of the bifunctional monomers (II) are in prepolymer form.

By way of example of monomer (III), there may be mentioned:
aromatic or aliphatic monoamines, such as dodecylamine, octadecylamine, benzylamine and the like,
aromatic or aliphatic monoacids containing from 1 to 32 carbon atoms, such as benzoic acid, acetic acid, propionic acid, saturated or unsaturated fatty acids (dodecanoic, oleic, palmitic or stearic acid and the like),
monofunctional alcohols or epoxides, such as ethylene oxide, epichlorohydrin and the like,
isocyanates such as phenyl isocyanate and the like,
diprimary diamines, which are preferably linear or branched, saturated aliphatic, having from 6 to 36 carbon atoms, such as, for example, hexamethylene-diamine, trimethylhexamethylenediamine, tetramethylene-diamine, n-xylenediamine,
saturated aliphatic dicarboxylic acids having from 6 to 36 carbon atoms such as, for example, adipic acid, azelaic acid, sebacic acid, maleic acid or anhydride,
difunctional alcohols or epoxides, such as ethylene glycol, diethylene glycol, pentanediol, glycidyl ethers of monofunctional alcohols containing from 1 to 24 carbon atoms,
diisocyanates, such as toluene diisocyanates, hexamethylene diisocyanate, phenyl diisocyanate, isophorone diisocyanate,
aromatic or aliphatic triamines, triacids or polyacids, triols or polyols such as N,N,N-tris(2-aminoethyl)amine, melamine and the like, citric acid, 1,3,5-benzenetricarboxylic acid and the like, 2,2,6,6-tetra(β-carboxyethyl)cyclohexanone, trimethylolpropane, glycerol, pentaerythritol, glycidyl ethers of di-, tri- or polyfunctional alcohols,
polymeric compounds such as poly- or monoamino polyoxyalkylenes marked under the trade mark JEFFAMINE®,
amino polyorganosiloxanes, such as amino polydimethylsiloxane.

The preferred “core” monomers (III) are: hexamethylenediamine, adipic acid, JEFFAMINE® T403 marketed by the company Huntsman, 1,3,5-benzene-tricarboxylic acid, 2,2,6,6-tetra(β-carboxyethyl)cyclohexanone.

By way of examples, the monomers (IV), there may be mentioned:
aromatic or aliphatic monoamines, such as dodecylamine, octadecylamine, benzylamine. Most of these compounds are generally considered as hydrophobic.
aromatic or aliphatic monoacids containing from 1 to 32 carbon atoms, such as benzoic acid, acetic acid, propionic acid, saturated or unsaturated fatty acids (dodecanoic, oleic, palmitic or stearic acid and the like). Most of these compounds are generally considered as hydrophobic.
monofunctional alcohols or epoxides, such as ethylene oxide, epichlorohydrin. Most of these compounds are generally considered as hydrophobic.
isocyanates such as phenyl isocyanate. Most of these compounds are generally considered as hydrophobic.
polymeric compounds such as monoamino polyoxyalkylenes, for example marketed under the trade mark JEFFAMINE M®, such as JEFFAMINE M 1000® and JEFFAMINE M 2070®. Most of these compounds are generally considered as hydrophilic.
monoamino silicone chains, such as monoamino polydimethylsiloxane. Most of these compounds are generally considered as hydrophobic.
N,N-dimethylaminopropylamine (hydrophilic or potentially hydrophilic, because it is basic or quaternizable for example with dimethyl sulfate).
N,N-diethylaminopropylamine (hydrophilic or potentially hydrophilic, because it is basic or quaternizable for example with dimethyl sulfate).
N,N-dibutylaminopropylamine (hydrophilic or potentially hydrophilic, because it is basic or quaternizable for example with dimethyl sulfate).
N-(3-aminopropyl)morpholine (hydrophilic or potentially hydrophilic, because it is basic or quaternizable for example with dimethyl sulfate).
N-methyl-N′-(3-aminopropyl)piperazine (hydrophilic or potentially hydrophilic, because it is basic or quaternizable for example with dimethyl sulfate).
N-(3-aminopropyl)piperidine (hydrophilic or potentially hydrophilic, because it is basic or quaternizable for example with dimethyl sulfate).
mixtures of these compounds.

Among the functional groups which may be present in the monomers (I) to (IV), and which are not capable of reacting with the functional groups A, A′, A″, B, B′ and B″, there may be mentioned in particular functional groups capable of providing or improving the hydrophilicity of the dendritic polymers used according to the invention. By way of example, there may be mentioned the quaternary ammonium, nitrile, sulfonate, phosphonate, phosphate, hydroxyl, polyethylene oxide, ether and (basic or quaternizable) ternary amine functional groups.

There may be mentioned:
4-aminobenzenesulfonic acid and its ammonium or alkali metal, in particular sodium, salts [monomer of formula (II)],
5-sulfosalicylic acid [monomer of formula (II)],
D- or L-2-amino-5-phosphorovaleric acid [monomer of formula (II)],
sulfobenzoic acid and its ammonium or alkali metal salts [monomer of formula (III) or (IV)],
epoxypropyltrimethylammonium chloride [monomer of formula (III) or (IV)],
polyethylene glycol polytioxyl,
aminomethylphosphonic acid [monomer of formula (IV)].

The hydrophilic functional groups may in particular be carried by the monomer (IV), for example by one of the following monomers:
polymeric compounds such as the monoamino polyoxyalkylenes for example marketed under the trade mark JEFFAMINE M®, such as JEFFAMINE M 1000® and JEFFAMINE M 2070®. Most of these compounds are generally considered as hydrophilic.
N,N-dimethylaminopropylamine (hydrophilic or potentially hydrophilic, because it is basic or quaternizable for example with dimethyl sulfate).
N,N-diethylaminopropylamine (hydrophilic or potentially hydrophilic, because it is basic or quaternizable for example with dimethyl sulfate).
N,N-dibutylaminopropylamine (hydrophilic or potentially hydrophilic, because it is basic or quaternizable for example with dimethyl sulfate).
N-(3-aminopropyl)morpholine (hydrophilic or potentially hydrophilic, because it is basic or quaternizable for example with dimethyl sulfate).
N-methyl-N′-(3-aminopropyl)piperazine (hydrophilic or potentially hydrophilic, because it is basic or quaternizable for example with dimethyl sulfate).
N-(3-aminopropyl)piperidine (hydrophilic or potentially hydrophilic, because it is basic or quaternizable for example with dimethyl sulfate).

Finally, the dendritic polymer may carry at the polymer chain ends a mixture of hydrophilic groups and hydrophobic groups, for example provided by monomers (IV) and/or acid-base control. It is thus possible to modulate the emulsifying properties and, where appropriate, make the action of the dendritic polymer sensitive to external conditions which can trigger stabilization or destabilization of the emulsion. This mode is preferable in the context of the preparation of multiple emulsions. There may be mentioned for example a combination of —COOH or COO⁻ groups and alkyl groups.

The dendritic polymers described above may be assimilated with arborescent structures endowed with a focal point formed by the functional group A and with a periphery provided with B ends. It is specified that the fact that the periphery is provided with B ends does not make it impossible for the B ends to be present at chain ends located further in the center of the dendritic polymer.

Moreover, when they are present, the bifunctional monomers (II) are spacer components in the three-dimensional structure. They make it possible to control the branching density.

When they are present, the monomers (III) form nuclei. The “chain limiting” monofunctional monomers (IV) are located at the periphery of the dendrimers. It should be specified that the fact that the periphery is provided with monofunctional monomers (IV) does not make it impossible for the monofunctional monomers (IV) to be present at chain ends located further in the center of the dendritic polymer.

The presence of monomers (III) and (IV) makes it possible in particular to control the molecular weight.

Preferably, the dendritic polymers used according to the invention are hyperbranched polyamides; they are obtained from at least one monomer of formula (I) having, as reactive polycondensation functional groups, amino functional groups, and carboxyl antagonist functional groups, or from a monomer composition additionally containing at least one monomer of formula (II) and/or (III) and/or (IV) having the same type(s) of reactive polycondensation functional group(s), it being possible for all or some of the monomer(s) of formula (II) to be replaced by a lactam.

The polycondensation/polymerization operation may be carried out in a known manner in a molten or solvent phase, it being possible for the monomer of formula (II), when it is present, to favorably play the role of solvent.

The operation may be favorably carried out in the presence of at least one polycondensation catalyst and optionally of at least one antioxidant compound. Such catalysts and antioxidant compounds are known to a person skilled in the art. By way of example of catalysts, there may be mentioned phosphorus compounds such as phosphoric acid, phosphorous acid, hypophosphorous acid, phenylphosphonic acids, such as 2-(2′-pyridyl)ethylphosphonic acid, phosphites such as tris(2,4-di-tert-butylphenyl)phosphite. By way of example of antioxidant, there may be mentioned di-hindered phenolic-based antioxidants such as N,N′-hexamethylenebis(3,5-di-tert-butyl-4-hydroxyhydrocinnamamide) or 5-tert-butyl-4-hydroxy-2-methylphenyl sulfate.

Hyperbranched polyamides having hydrophilic functionalities which are not reactive with the functional groups A, A′, A″, B, B′ and B″ may be obtained using a monomer of formula (III) and/or (IV) having one or more polyoxyethylene groups (for example a monomer of the JEFFAMINES amino polyoxyalkylene family) and/or a monomer of formula (IV) having quaternary ammonium, nitrile, sulfonate, phosphonate or phosphate functional groups.

Another embodiment consists, after preparation of a hyperbranched polymer by polycondensation of nonfunctional monomers, in modifying the terminal functional groups of said hyperbranched polyamide by reaction with a compound having hydrophilic functional groups. This may be for example a compound having a tertiary amine, quaternary ammonium, nitrile, sulfonate, phosphonate or phosphate group or polyoxyethylene groups. The terminal functional groups may also be modified by a simple acid-base type reaction, by completely or partially ionizing the groups included at the chain ends. For example, terminal groups of the carboxylic acid type (for example B, B′ and/or B″ groups), may be made anionic by adding a base. Terminal groups of the amine type (for example B, B′ and/or B″ groups) may be made cationic by adding an acid.

It should be noted that the functionalization may be complete or partial. It is preferably greater than 25% in numerical terms, relative to the entire free functional groups carried (B, B′, B″).

It should be noted that it is not impossible to carry out a hydrophobic partial functionalization after preparing the dendritic polymer. It is thus possible to modulate the emulsifying properties and, where appropriate, make the action of the dendritic polymer sensitive to external conditions which may trigger stabilization or destabilization of the emulsion.

The weight-average molar mass of said dendritic, in particular hyperbranched polyamide, polymers may range from 500 to 1 000 000 g/mol, preferably from 1000 to 500 000 g/mol, more preferably still from 3000 to 20 000 g/mol.

The weight-average molar mass may be measured by size exclusion chromatography. The measurement is carried out in an eluent phase composed of 70% by volume of 18 megaohm Millipore water and 30% by volume of methanol, containing 0.1M NaNO₃; it is adjusted to pH 10 (1/1000 NH₄OH 25%).

The weight-average molar mass is established in a known manner by means of light scattering values.

Quantities—Formulation

The ratio by weight between the quantities of inner phase and outer phase is preferably between 0.1/99.9 and 95/5, more preferably between 1/99 and 10/90.

The weight ratio between the quantities of dendritic polymer and inner phase is preferably between 0.05/100 and 20/100, more preferably between 0.5 and 20/100 or even between 5/100 and 20/100.

Moreover, the proportion by weight of dendritic polymer in the whole emulsion is preferably between 0.05% and 10%, more preferably still between 0.1% and 5%, for example of the order of 1%.

The size of the emulsion droplets may depend on the quantity of emulsifying agent (dendritic polymer optionally with other agents such as surfactants) used and/or the amount of energy used to prepare the emulsion. At a low proportion of emulsifying agent, the size of the droplets may be mostly limited (lower limit, large size droplets) by the quantity of emulsifying agent. The higher the proportion of emulsifying agent, the smaller the droplets. Reference is then often made to a poor regime. At a higher proportion of emulsifying agent, the size may be mostly limited (lower limit) by the amount of energy. The higher the amount of energy, the smaller the droplets. Reference is often made to a rich regime. In the case where the emulsion comprises no other emulsifying agent than the dendritic polymer, the limit between the poor regime and the rich regime may be of the order of a few %, for example 1/100 to 2/100 (ratio by weight between the quantities of dendritic polymer and of inner phase), for a direct emulsion.

It may be mentioned, with no limitation to the invention being implied, that it has been observed that the critical concentration (by weight of dendritic polymer) between the poor regime and the rich regime does not appear to depend on the molar mass of the dendritic polymer. Without wishing to be bound by any theory, it is thought that the dendritic polymer is present at the interface between the aqueous phase and the hydrophobic phase, in the form of aggregated objects around the droplets.

Thus, it is possible to operate such that the size of the droplets is modulated by acting on the nature of the inner phase, the proportion of the various constituents, in particular the emulsifying agent, and on process parameters (rate and duration of mixing to confer energy).

Other Ingredients

The emulsions according to the invention are compositions which, in addition to the ingredients mentioned above, may comprise other ingredients. The nature and the quantity of these other ingredients may depend on the destination or use of the emulsion. These additional ingredients are known to a person skilled in the art.

For example, the emulsion may comprise additional known emulsifying agents in combination with the dendritic polymer, in particular surfactants, in particular nonionic or cationic surfactants, water-soluble amphiphilic polymers, comb polymers or block polymers.

In the context of multiple emulsions, it is specified that each of the aqueous phases may comprise agents intended to control the osmotic pressure. This may. be for example a salt chosen from alkali or alkaline-earth metal halides (such as sodium chloride, calcium chloride), or a sugar (such as glucose) or a polysaccharide (such as dextran), or a mixture.

In general, the emulsions may comprise nonionic, anionic, cationic or amphoteric surfactants (zwitterionic surfactants being included among the amphoteric surfactants).

The emulsions may also comprise pH-regulating agents, active substances, perfumes and the like.

Process

The emulsions according to the invention may be prepared by conventional emulsifying processes. These processes conventionally consist in more or less vigorously mixing the various ingredients: the immiscible phases, the emulsifying agent and optionally other ingredients. For this mixture, some of the ingredients may have been mixed, dissolved or dispersed beforehand. Thus, it may be advantageous to use an aqueous phase into which the dendritic phase has been introduced beforehand, before mixing said aqueous phase with the immiscible phase.

The mixture may be prepared with more or less vigorous stirring. In the case where the inner phase is not very viscous (viscosity less than 1 Pa·s), the procedure may be advantageously carried out with vigorous stirring, for example with the aid of a Microfluidizer Ultra-turrax® type apparatus, or any other high-pressure homogenizer. In the case where the inner phase is viscous (viscosity greater than 1 Pa·s, preferably greater than 5 Pa·s), the procedure may be advantageously carried out with the aid of a paddle frame.

The temperature at which the emulsion is prepared may depend on the various phases used. Thus, it is possible to choose to modulate the temperature in order to modulate the viscosity of the various phases used. It should be noted that it may be practical to add a thermothickening compound to the inner phase.

The duration of stirring may be determined with no difficulty by a person skilled in the art. It generally depends on the apparatus used. In a rich regime, it may partly determine the size of the droplets.

It should also be mentioned that the emulsions may be prepared according to a self-emulsifying process. Under certain conditions, a mixture comprising the compound which will constitute the inner phase and the emulsifying agent(s) can form an emulsion by simple addition to water, with very gentle stirring. Reference is made, for this mixture, to self-emulsifiable compositions. Such compositions find use in particular in the agricultural field, to formulate water-insoluble liquid plant-protection compounds directly on the farm (tank mix), and in the field of coatings and paints (in particular for isocyanate bases).

Applications

The emulsions according to the invention may be used in numerous fields of application. There may be mentioned most particularly the fields of formulation of cosmetic products (skin or hair care, makeup), of detergent products (cleaning of linen, dishes or hard surfaces), of paints or of coatings.

In the detergent or cosmetic field, the dendritic polymer according to the invention can serve as emulsion vector or as trigger for depositing on a surface a compound in the form of an emulsion, for example a silicone. Thus, a stable emulsion of a compound to be deposited (for example a silicone) is prepared and the deposition is triggered by modifying the outer phase, for example by dilution or by change of pH, so as to modify the hydrophilicity of the groups contained in the dendritic polymer (modification to make them more hydrophobic). The emulsion may then be destabilized, and the emulsified compound becomes deposited on a surface, for example a textile surface (detergency), or on the skin or hair (cosmetic, conditioning effect). The emulsified compound may also be brought to the surface by simple affinity of the dendritic polymer for the surface, by adsorption for example.

Regardless of the mechanism, destabilization of the emulsion or affinity for a surface, the dendritic polymer can be considered as an emulsion vector. It is particularly useful in shampoos or in textile care compositions. These mechanisms may also be used for depositions or treatments on metals, glass or clays.

In the cosmetic field, the emulsions have the advantage of being substantially free of surfactant and of not foaming in the absence of a surfactant. The dendritic polymer may be combined with a surfactant. In this case, the dendritic polymer has an effect on the emulsification, without increasing the foaming linked to the presence of surfactant. In addition, in the absence of a surfactant, the dendritic polymer does not foam, and in the presence of a surfactant that is not very foamy, it improves the emulsifying or emulsion stability properties, without increasing foaming. Completely avoiding foaming, or not increasing it, avoids using constraining emulsifying processes. Moreover, some products are not intended to foam. These are in general creams, milks or gels intended to be applied to the skin or to the lips.

As regards the field of paints and coatings, the emulsions according to the invention may for example be emulsions of the alkyd or isocyanate type (emulsion of an alkyd or an isocyanate in water). The emulsion may also be an emulsion of monomers intended for the preparation of latex.

The emulsion according to the invention may be used in paints, preferably aqueous paints, or may itself constitute a paint, preferably an aqueous paint, and may be used to transport in particular a hydrophobizing agent on a surface of the construction material, plaster, cement or wood type and the like, with release of the hydrophobizing agent by depositing and drying the paint on the surface.

It can also be used for the treatment of metals.

Likewise, it can be used in cosmetic compositions or can itself constitute an aqueous cosmetic composition (moisturizing creams, antisun creams, makeup products, hair styling gels and the like); the hydrophobic phase may be or may contain any hydrophobic care active substance (such as conditioning agents, disentangling agents and the like), anti-UV agents, pigments, colorants and the like.

It can also be used to confer on surfaces made of woven or unwoven material of cellulosic and/or synthetic origin, for body hygiene or household cleaning, intended to be brought into contact with the skin, such as care, cleansing or makeup-removing wipes, absorbent tissues, feminine protection (towels), diapers and the like, benefits intrinsic to the hydrophobic nonaqueous phase and/or to active substances contained in the hydrophobic phase, this being during the preparation of said surfaces or by post-treatment of said surfaces. Softening, anti-odor, perfuming and bactericidal properties and the like may thus be conferred.

It may also be used during the manufacture or for the post-treatment of cartons or carton packagings, to provide hydrophobic, anti-odor, bactericidal and fragrant properties and the like.

The emulsion according to the invention (E) is particularly advantageous for transporting and depositing a hydrophobic acid substance (constituting the hydrophobic phase or contained in the hydrophobic phase) on a surface or a substrate (S) made of hydroxyapatite (tooth), a keratin surface or substrate (skin, hair, leather) or a textile surface or substrate.

When said substrate (S) is made of hydroxyapatite (teeth), the hydrophobic phase may contain hydrophobic agents having refreshing properties, agents which make it possible to combat dental plaque, antiseptic agents and the like. The emulsion (E) may be contained in or can itself form a composition for dental or oral hygiene, a composition intended to be rinsed out or diluted. This may be toothpastes, mouthwashes and the like.

Said substrate (S) may be in particular a keratin surface such as the skin and the hair. The hydrophobic phase may be or may contain any hydrophobic care active substance (such as conditioning agents, disentangling agents and the like), anti-UV agents, pigments, colorants and the like; the emulsion (E) may be contained in or may itself form a cosmetic composition intended to be rinsed off or diluted; this may be in particular a shampoo, a conditioner, a shower gel and the like.

The said substrate (S) may be leather; the hydrophobic phase may be or may contain any hydrophobic active substance capable of providing softness, suppleness and protection against external agents, and the like, to the hydrophobic substrate.

Advantageously, said substrate (S) is a textile material.

The textile substrate may be provided in the form of textile fibers or articles made from natural textile fibers (cotton, flax or other natural cellulosic material, wool and the like), artificial fibers (viscose, rayon and the like) or synthetic fibers (polyamide, polyester and the like) or mixtures thereof.

Preferably, said substrate is a textile surface made of a cellulosic material, of cotton in particular.

The hydrophobic phase is preferably made of a textile care agent.

The benefits provided by a lubricating hydrophobic phase to a textile substrate are in particular the provision of properties of softness, anti-wrinkling, easy-ironing, abrasion resistance (protection in particular against aging when wearing the clothing or during repeated washing operations), elasticity, protection of the colors, retention of fragrances and the like.

Among the other active substances providing other benefits in the field of the care of articles made of textile fibers, there may be mentioned in particular fragrances; preferably, these are dissolved in the hydrophobic phase.

The substrate or the surface (S) may be present in an aqueous bath (B). The aqueous bath (B) in which the textile substrate is present to acquire benefits therein may be highly varied. This may be, without limitation, a bath for soaking, washing, rinsing or padding, and the like.

The emulsion according to the invention may be used in particular as additive in a detergent composition for washing or rinsing articles made of textile fibers, or as a detergent or rinsing composition for washing or rinsing articles made of textile fibers, with the aim of transporting a hydrophobic textile care agent and/or any other useful hydrophobic active substance, and of promoting the deposition thereof on an article made of textile fibers, of cotton in particular, during the rinsing operation and/or during the drying operation subsequent to the main washing operation in the case of a detergent composition for washing, or during the subsequent drying operation in the case of a rinsing composition.

The emulsion in the form of a multiple emulsion containing a care hydrophobic phase, as detergent composition or in a detergent composition for washing linen in a washing machine, used during the washing cycle, and without adding a softening rinsing liquid during the rinsing cycle, made it possible to give the washed linen properties of softness, suppleness, anti-wrinkling, easy-ironing, resistance to abrasion, elasticity, protection of the colors, retention of fragrances, and the like.

The emulsion (E) in the form of a multiple emulsion containing a care hydrophobic phase, as rinsing composition or in a composition for rinsing linen, makes it possible to give the linen, after drying, properties of softness, suppleness, anti-wrinkling, easy-ironing, resistance to abrasion, elasticity, protection of the colors, retention of fragrances, and the like.

The deposition of the hydrophobic phase containing or consisting of an active substance (A) on the substrate may be by deposition by adsorption, cocrystallization, trapping and/or adhesion.

The quantity of emulsion in the form of a multiple emulsion which may be present in a composition for washing articles made of textile fibers, according to the third subject of the invention, corresponds to a quantity of hydrophobic phase representing from 0.0001% to 25%, preferably from 0.0001% to 5% of the total weight of the composition, with relative quantities of emulsion, expressed as multiple emulsion, and of aqueous medium (B) which are equivalent to a 2 to 100-fold dilution of the volume of said emulsion.

The quantity of emulsion in the form of a multiple emulsion which may be present in a composition for rinsing articles made of textile fibers, according to the third subject of the invention, corresponds to a quantity of hydrophobic phase representing from 0.0001% to 25%, preferably from 0.0001% to 5% of the total weight of the composition, with relative quantities of emulsion, expressed as multiple emulsion, and of aqueous medium (B) which are equivalent to a 2 to 100-fold dilution of the volume of said emulsion.

A washing composition made of compacted or noncompacted powder, or in liquid form, for articles made of textile fibers may contain at least one surfactant preferably chosen from anionic and nonionic surfactants or mixtures thereof.

Among the anionic surfactants, there may be mentioned (C₈-C₁₅)alkylbenzenesulfonates (in an amount of 0-30%, preferably 1-25%, more preferably 2-15% by weight).

In addition, there may be mentioned primary or secondary alkyl sulfates, in particular primary (C₈-C₁₅)alkyl sulfates; alkyl ether sulfates; olefin sulfonates; alkylxylene sulfonates; dialkyl sulfosuccinates; sulfonate esters of fatty acids; the sodium salts are generally preferred.

Among the nonionic surfactants, there may be mentioned primary or secondary alcohol ethoxylates, in particular aliphatic C₈-C₂₀ alcohol ethoxylates having from 1 to 20 moles of ethylene oxide per mole of alcohol, and more particularly primary or secondary aliphatic C₁₀-C₁₅ alcohol ethoxylates having from 1 to 10 moles of ethylene oxide per mole of alcohol; there may also be mentioned nonethoxylated nonionic surfactants such as alkyl polyglucosides, glycerol monoethers and polyhydroxyamides (glucamides).

Preferably, the nonionic surfactant level is 0-30%, preferably 1-25%, more preferably 2-15% by weight.

The choice and the quantity of surfactant depend on the desired use of the detergent composition. The surfactant systems to choose for washing textiles by hand or by machine are well known to formulators.

Quantities of surfactants as high as 60% by weight may be present in the compositions for washing by hand. Quantities of 5-40% by weight are generally suitable for washing textiles by machine. Typically, these compositions comprise at least 2% by weight, preferably 2-60%, more preferably 15-40% and particularly 25-35% by weight.

It is also possible to include cationic monoalkyl surfactants. There may be mentioned the quaternary ammonium salts of formula R¹R²R³R⁴N⁺X⁻ where the groups R are long or short hydrocarbon chains, alkyl chains, hydroalkyl chains or ethoxylated alkyl chains, X being a counterion (R¹ is a C₈-C₂₂, preferably C₈-C₁₀ or C₁₂-C₁₄, alkyl group and R² is a methyl group, R³ and R⁴, which are similar or different, being a methyl or hydroxymethyl group); and cationic esters, such as choline esters.

The detergent compositions for most washing machines generally contain an anionic surfactant different from soaps, or a nonionic surfactant, or mixtures thereof, and optionally a soap.

The detergent compositions for washing textiles generally contain at least one builder; the total quantity of builder is typically 5-80%, preferably 10-60% by weight.

There may be mentioned inorganic builders such as sodium carbonate, crystalline or amorphous aluminosilicates (10-70%, preferably 25-50% on a dry basis), laminar silicates, inorganic phosphates (Na orthophosphate, pyrophosphate and tripolyphosphate). Further details relating to particularly suitable aluminosilicates and zeolites are given in WO 03/020819.

There may also be mentioned organic builders such as polymers of the polyacrylate type, acrylic/maleic copolymers and acrylic phosphinates; monomeric polycarboxylates such as glycerol citrates, gluconates, oxidisuccinates, mono-, di- and trisuccinates, alkyl or alkenyl dipicolinates, hydroxyethyliminodiacetates, malonates or succinates; sulfonated fatty acid salts and the like.

Preferably, the organic builders are citrates (5-30%, preferably 10-20% by weight), acrylic polymers, more particularly acrylic/maleic copolymers (0.5-10%, preferably 1-10% by weight).

When they are in compacted or noncompacted powdered form, the compositions may favorably contain a bleaching system, in particular peroxide compounds such as inorganic persalts (perborates, percarbonates, perphosphates, persilicates and persulfates, preferably sodium perborate monohydrate or tetrahydrate, and sodium percarbonate) or organic peroxy acids (urea peroxide), which are capable of releasing oxygen in solution.

The bleaching peroxide compound is favorably present in an amount of 0.1-35%, preferably 0.5-25% by weight. It may be combined with a bleaching activator in order to improve bleaching at low temperature; it is favorably present in a quantity of 0.1-8%, preferably of 0.5-5% by weight. The preferred activators are peroxycarboxylic acids, in particular peracetic and pernonanoic acids. There may be mentioned most particularly N,N,N′,N′-tetraacetylethylenediamine (TAED) and sodium nonanoyloxybenzenesulfonate (SNOBS).

The compositions generally also comprise one or more enzymes, in particular proteases, amylases, cellulases, oxidases, peroxidases and lipases (0.1-3% by weight), fragrances, anti-redeposition agents, antisoiling agents, anti-color transfer agents and nonionic softeners, and the like.

The detergent compositions for washing textiles may also be provided in the form of nonaqueous liquid bars in an envelope made of a material which becomes dispersed in the laundry detergent medium such as polyvinyl alcohol for example.

They comprise at least one water-miscible alcohol such as in particular isopropyl alcohol, in a quantity which may range from 5 to 20% by weight.

They may contain at least one surfactant preferably chosen from anionic and nonionic surfactants or mixtures thereof, in a quantity which may range from 20 to 75% by weight.

They may additionally comprise organic builders such as sodium citrates; phosphonates and the like, in a quantity which may range from 5 to 20% by weight; they may also comprise fragrances, colorants and the like.

The compositions for rinsing articles made of textile fibers may contain cationic or nonionic softeners. They may represent from 0.5 to 35%, preferably 1-30%, more preferably 3-25% of the weight of the rinsing composition.

Cationic softeners are substantially non-water-soluble quaternary ammonium compounds comprising a single alkyl or alkenyl chain containing at least 20 carbon atoms, or preferably compounds having two polar heads and two alkyl or alkenyl chains containing at least 14 carbon atoms. Most preferably, the softening compounds have two alkyl or alkenyl chains containing at least 16 carbon atoms, and particularly at least 50% of the alkyl or alkenyl groups have 18 carbon atoms or more. Most preferably, the linear alkyl or alkenyl chains are predominant.

In commercial rinsing softening formulas, quaternary ammonium compounds are very commonly used which have two long aliphatic chains, such as distearydimethylammonium and ditallowalkyldimethylammonium chlorides.

The rinsing compositions may additionally comprise nonionic softeners such as lanolin; lecithins and other phospholipids are also suitable. The rinsing compositions may also contain nonionic stabilizing agents such as alkoxylated linear C₈-C₂₂ alcohols containing 10 to 20 moles of alkylene oxide, C₁₀-C₂₀ alcohols and mixtures thereof. The quantity of nonionic stabilizing agent represents from 0.1 to 10%, preferably 0.5-5% and most particularly 1-4% of the weight of the composition. The molar ratio of the quaternary ammonium compound and/or other cationic softener to the stabilizing agent is favorably 40/1-1/1, preferably 18/1-3/1.

The composition may additionally comprise fatty acids, in particular (C₈-C₂₄) alkyl- or alkenylmonocarboxylic acids or polymers thereof; preferably they are saturated and nonsaponified, such as oleic, lauric or tallow acids. They may be used in an amount of at least 0.1%, preferably of at least 0.2% by weight. In concentrated compositions, they may be present in an amount of 0.5-20%, preferably 1-10% by weight. The molar ratio of the quaternary ammonium compound and/or other cationic softener to the fatty acid is favorably 10/1-1/10.

Other details or advantages of the invention will emerge in the light of the examples below, with no limitation being implied.

### EXAMPLE 1

synthesis of a hyperbranched copolyamide with carboxylic acid ends by molten phase copolycondensation of 1,3,5-benzenetricarboxylic acid (noted BTC, a core molecule of the R¹—B″₃ type, with B″═COOH), of 5-aminoisophthalic acid (noted AIPA, a branching molecule of the A-R—B₂ type, with A=NH₂ and B═COOH), and of ε-caprolactam (noted CL, a spacer of the A′-R′-B′ type, with A′=NH₂ and B′═COOH). The respective overall composition is 1/6/6 as BTC/AIPA/CL.

The reaction is carried out in a 500 ml glass reactor commonly used in the laboratory for the molten phase synthesis of polyesters or polyamides.

The monomers are completely loaded at the beginning of the trial. The reactor is immersed in a Wood's alloy metal bath at 100° C. and kept mechanically stirred at 80 rpm. 72.7 g of ε-caprolactam (0.64 mol), 116.4 g of 5-aminoisophthalic acid (0.64 mol), 22.5 g of 1,3,5-benzenetricarboxylic acid (0.11 mol) and 0.53 g of a 50% (w/w) aqueous hypophosphorous acid solution are successively introduced into the reactor. The reactor is placed under a weak current of dry nitrogen.

The stirring is then set at 50 rpm and the reaction mass is gradually heated from 100° C. to 250° C., in about 250 min. The temperature is then maintained at 250° C. as a plateau.

After 60 minutes under these conditions, the reactor is gradually placed under a vacuum over 60 min. The minimum vacuum is then maintained for an additional 60 min. About 10.6 g of distillate are recovered.

At the end of the cycle, the stirring is stopped and the reactor is allowed to cool to room temperature under a nitrogen stream. 182.5 g of polymer are recovered.

The hyperbranched copolyamide obtained is a whitish solid.

### EXAMPLE 2

synthesis of a hyperbranched copolyamide with carboxylic acid ends by molten phase copolycondensation of 1,3,5-benzenetricarboxylic acid (noted BTC, a core molecule of the R¹—B″₃ type, with B″═COOH), of 5-aminoisophthalic acid (noted AIPA, a branching molecule of the A-R—B₂ type, with A=NH₂ and B═COOH), and of ε-caprolactam (noted CL, a spacer of the A′-R′—B′ type, with A′=NH₂ and B′═COOH). The respective overall composition is 1/25/25 as BTC/AIPA/CL.

The same reactor as that described in example 1 is used. The monomers are completely loaded at the beginning of the trial. The reactor is immersed in a Wood's alloy metal bath at 100° C. and kept mechanically stirred at 80 rpm.

79.5 g of ε-caprolactam (0.70 mol), 127.2 g of 5-aminoisophthalic acid (0.70 mol), 5.9 g of 1,3,5-benzenetricarboxylic acid (0.03 mol) and 0.49 g of a 50% (w/w) aqueous hypophosphorous acid solution are successively introduced into the reactor. The reactor is placed under a weak current of dry nitrogen.

The stirring is then set at 50 rpm and the reaction mass is gradually heated from 100° C. to 250° C., in about 250 min. The temperature is then maintained at 250° C. as a plateau.

After 60 minutes under these conditions, the reactor is gradually placed under a vacuum over 60 min. The minimum vacuum is then maintained for an additional 60 min. About 11.3 g of distillate are recovered.

At the end of the cycle, the stirring is stopped and the reactor is allowed to cool to room temperature under a nitrogen stream. 162.2 g of polymer are recovered.

The hyperbranched copolyamide obtained is a whitish solid.

### EXAMPLE 3

Neutralization with sodium hydroxide of a hyperbranched copolyamide with carboxylic acid ends having an overall composition 1/25/25 respectively as BTC/AIPA/CL, synthesized in example 2.

50.0 g of hyperbranched copolyamide obtained in example 2 are finely ground and dispersed in 300 ml of water. The mixture is mechanically stirred with the aid of an anchor and gradually supplemented with 35% by mass of aqueous sodium hydroxide. The pH is regularly checked with the aid of pH paper and maintained around 10. 22.12 g of sodium hydroxide are required to reach a stable pH. The solution is then filtered and then freeze-dried. 48.8 g of fine white powder are recovered.

Elemental analysis of sodium gives an average content of 9% by mass, that is a content of sodium carboxylate groups of 3480 meq/kg.

### EXAMPLE 4

synthesis of a hyperbranched copolyamide with polyalkylene oxide ends by molten phase copolycondensation of 1,3,5-benzenetricarboxylic acid (noted BTC, a core molecule of the R¹—B″₃ type, with B″═COOH), of 5-aminoisophthalic acid (noted AIPA, a branching molecule of the A-R—B₂ type, with A=NH₂ and B═COOH), of ε-caprolactam (noted CL, a spacer of the A′-R′-B′ type, with A′=NH₂ and B′═COOH) and of Jeffamine M1000® (noted M1000, blocker of the A″-R² type, with A″=NH₂). The respective overall composition is 1/25/25/28 as BTC/AIPA/CL/M1000.

The same reactor as that described in example 1 is used. The monomers are completely loaded at the beginning of the trial. The reactor is immersed in a Wood's alloy metal bath at 100° C. and kept mechanically stirred at 80 rpm.

23.9 g of ε-caprolactam (0.21 mol), 236.2 g of Jeffamine M1000® (0.24 mol), 38.2 g of 5-aminoisophthalic acid (0.21 mol), 1.8 g of 1,3,5-benzenetricarboxylic acid (0.008 mol) and 0.22 g of a 50% (w/w) aqueous hypophosphorous acid solution are successively introduced into the reactor. The reactor is placed under a weak current of dry nitrogen.

The stirring is then set at 50 rpm and the reaction mass is gradually heated from 100° C. to 250° C., in about 250 min. The temperature is then maintained at 250° C. as a plateau.

After 60 minutes under these conditions, the reactor is gradually placed under a vacuum over 60 min. The minimum vacuum is then maintained for an additional 60 min. About 7.0 g of distillate are recovered.

At the end of the cycle, the stirring is stopped and the reactor is allowed to cool to room temperature under a nitrogen stream. 281.5 g of polymer are recovered.

The hyperbranched copolyamide obtained is a translucent viscous liquid.

### EXAMPLE 5

synthesis of a hyperbranched copolyamide with polyalkylene oxide and carboxylic acid ends by molten phase copolycondensation of 1,3,5-benzenetricarboxylic acid (noted BTC, a core molecule of the R¹—B″₃ type, with B″═COOH), of 5-aminoisophthalic acid (noted AIPA, a branching molecule of the A-R—B₂ type, with A=NH₂ and B═COOH), of ε-caprolactam (noted CL, a spacer of the A′-R′-B′ type, with A′=NH₂ and B′═COOH) and of Jeffamine M1000® (noted M1000, blocker of the A″-R² type, with A″=NH₂). The respective overall composition is 1/25/25/21 as BTC/AIPA/CL/M1000.

The same reactor as that described in example 1 is used. The monomers are. completely loaded at the beginning of the trial. The reactor is immersed in a Wood's alloy metal bath at 100° C. and kept mechanically stirred at 80 rpm.

29.3 g of ε-caprolactam (0.26 mol), 221.7 g of Jeffamine M1000® (0.22 mol), 46.9 g of 5-aminoisophthalic acid (0.26 mol), 2.2 g of 1,3,5-benzenetricarboxylic acid (0.010 mol) and 0.24 g of a 50% (w/w) aqueous hypophosphorous acid solution are successively introduced into the reactor. The reactor is placed under a weak current of dry nitrogen.

The stirring is then set at 50 rpm and the reaction mass is gradually heated from 100° C. to 250° C., in about 250 min. The temperature is then maintained at 250° C. as a plateau.

After 60 minutes under these conditions, the reactor is gradually placed under a vacuum over 60 min. The minimum vacuum is then maintained for an additional 60 min. About 11.9 g of distillate are recovered.

At the end of the cycle, the stirring is stopped and the reactor is allowed to cool to room temperature under a nitrogen stream. 285.8 g of polymer are recovered.

The hyperbranched copolyamide obtained is a translucent viscous liquid, which hardens into a wax at room temperature.

### EXAMPLES 6 TO 9

### Preparation of Water-in-Oil Direct Emulsions Containing 20% by Weight of Oily Phase and 80% of Aqueous Phase

Hyperbranched Copolyamides (HBPA) Prepared According to the preceding examples are used as emulsifying agent. The quantity of HBPA taken for the preparation of the emulsion is solubilized in water beforehand to prepare the aqueous phase. The latter is adjusted to a desired pH by addition of a 1N NaOH or HCl solution.

The oily phase is added to the aqueous phase with stirring with the aid of a rotor/stator type stirrer (Ultra-turrax) revolving at 9500 rpm. After addition, the stirring is extended for 2 min.

The emulsion thus obtained is then subjected to 3 runs at a pressure of 250 bar or 500 bar in a high-pressure homogenizer (MICROFLUIDIZER M110S).

The particle size distribution of the emulsion thus obtained is measured with a laser diffraction granulometer (HORIBA LA-910 granulometer) and the variation of this particle size distribution and the variation of the macroscopic stability of the emulsion are monitored over time in order to observe the instability phenomena which may occur (coalescence, Oswald ripening, creaming or sedimentation of the droplets due to the difference in densities between the oil and the water).

### EXAMPLE 6

### Influence of the Concentration of HBPA in the Aqueous Phase on the Size of the Emulsion

Emulsions comprising from 0.25 to 5% by weight of HBPA relative to the oil are prepared.

An HBPA according to example 1 and an HBPA according to example 2 are used. The pH of the aqueous phase is adjusted to 6.0-6.5.

The oily phase is hexadecane. The homogenization pressure is 500 bar.

The median radius (R) of the emulsion is measured as a function of the HBPA/oil concentration. It shows that the definition of the domains poor (P) and rich (R) in polymer and that the size of the emulsion for a given concentration of polymer are relatively independent of the molecular mass of the dendritic polymer.

The results are presented in table I below.

TABLE I

| HBPA | R (μm) with HBPA | R (μm) with HBPA |
|---|---|---|
| concentration (%) | according to example 1 | according to example 2 |
| 0.25 | | 0.79 |
| 0.50 | 0.67 | 0.53 |
| 1.0 | 0.37 | 0.30 |
| 2.0 | | 0.24 |
| 2.5 | 0.24 | |
| 5.0 | 0.23 | 0.23 |

### EXAMPLE 7

### Emulsions Prepared with an HBPA According to Example 2, and Various Oils

The emulsion contains 1% by weight of HBPA according to example 2 relative to the oil (that is 0.2% in the emulsion). The pH of the aqueous phase is adjusted to 6.0-6.5. 3 oils are studied: hexadecane, a silicone oil polydimethylsiloxane (Rhodorsil V100 from Rhodia) and a rape methyl ester (Phytorob 926-65 from Novance).

The emulsions are subjected to 3 runs at 200 bar in the Microfluidizer.

The results in terms of stability are presented in Table I below.

TABLE I

| | Age of the | Median | Macroscopic |
|---|---|---|---|
| Oil studied | emulsion | diameter in μm | stability |
| Hexadecane | 1 hour | 0.38 | stable |
| | 8 days | 0.40 | stable |
| Silicone oil | 1 hour | 0.53 | stable |
| | 8 days | 0.48 | stable |
| Rape ester | 1 hour | 0.28 | stable |
| | 8 days | 0.48* | stable |

*increase in the size of the drops due to Oswald ripening caused by the relatively high solubility of the rape ester in water.

### EXAMPLE 8

Emulsions prepared with HBPA according to examples 1 or 4. Influence of the nature of the chain ends.

The emulsions contain between 0.5 and 5% by weight of HBPA relative to the oil (0.1 to 1.0% in the emulsion).

The oil phase is hexadecane.

The emulsions are subjected to 3 runs at 200 bar in the Microfluidizer M110S (3 runs at 500 bar with HBPA with an amine end).

The results are presented in Table II below.

TABLE II

| | | Age of the | Median | Macroscopic |
|---|---|---|---|---|
| HBPA | %/hexadecane | emulsion | diameter | stability |
| example | 0.5% | 1 h | 0.58 | stable |
| 1 | | 8 d | 0.58 | slight creaming |
| | 2.0% | 1 h | 0.30 | stable |
| | | 8 d | 0.30 | stable |
| example | 1% | 1 h | 0.82 | stable |
| 4 | | 8 d | 0.86 | slight creaming |
| | 5% | 1 h | 0.33 | stable |
| | | 8 d | 0.33 | stable |

The creaming observed after 8 days is due to the large difference in density between the hexadecane and the water which causes the gradual rise of the larger droplets to the top part of the emulsion.

### EXAMPLE 9

Emulsions prepared with an HBPA according to example 1. Influence of the pH of the aqueous phase.

The emulsion contains 5% by weight of HBPA relative to the oil (that is 1% in the emulsion).

3 emulsions are prepared with an aqueous phase at three different pH values: 10.4-7.0-5.5. At pH 5.5, the polymer is at the solubility limit.

The oil used is a rape methyl ester (Phytorob 926-65 from Novance).

The emulsions are subjected to 3 runs at 200 bar in the Microfluidizer.

The results are presented in Table III below.

| pH of the | Age of the | | Median | |
|---|---|---|---|---|
| aqueous phase | emulsion | | diameter | Macroscopic stability |
| 10.4 | 1 | h | 0.27 | stable |
| | 24 | h | — | 5 to 7% |
| | 8 | d | — | coalescence total phase |
| | | | | separation |
| 7.0 | 1 | h | 0.28 | stable |
| | 24 | h | 0.32 | 1 to 2% coalescence |
| | 8 | d | | 10 to 12% coalescence |
| 5.5 | 1 | h | 0.28 | stable |
| | 24 | h | 0.33 | stable |
| | 8 | d | 0.44 | stable |

At pH 5.5, the polymer is at the solubility limit in water and it is in this pH region that its affinity for the water/oil interface is the highest, which explains the very good stability of the emulsions and the absence of coalescence. The solubility and the affinity of the polymer for water increases with the pH and brings about a lower stability of the interfaces and the development of increasingly great coalescence. The increase in the median diameter observed even at pH 5.5 is due to the Oswald ripening brought about by the solubility of the rape ester in water.

### EXAMPLE 10

Synthesis of a hyperbranched copolyamide with carboxylic acid and octadecylene ends by molten phase copolycondensation of 1,3,5-benzenetricarboxylic acid (noted BTC, a core molecule of the R¹—B″₃ type, with B″═COOH), of 5-aminoisophthalic acid (noted AIPA, a branching molecule of the A-R—B₂ type, with A=NH₂ and B═COOH), of ε-caprolactam (noted CL, a spacer of the A′-R′—B′ type, with A′=NH₂ and B′═COOH) and of octadecylamine (noted C18, a blocker of the A″-R₂ type, with A″=NH₂). The respective overall composition is 1/25/25/2 as BTC/AIPA/CL/C18.

The same reactor as that described in example 1 is used. A Wood's alloy metal bath is used for heating the reaction mixture.

74.3 g of ε-caprolactam (0.656 mol) and 66.4 g of demineralized water are introduced into the reactor at room temperature. After dissolution, 118.9 g of 5-aminoisophthalic acid (0.656 mol), 5.5 g of 1,3,5-benzenetricarboxylic acid (0.026 mol) and 0.476 g of a 50% (w/w) aqueous hypophosphorous acid solution are added. The reaction mixture is then mechanically stirred at 50 rpm. A weak current of dry nitrogen is produced and heating at 100° C. is triggered.

The reaction mass is then rapidly. heated from 100° C. to 165° C. in about 15 min. An isothermal plateau is produced at this temperature for 150 min.

After one hour of plateau, when the distillation of the water in the stock solution has been carried out, 14.1 g of octadecylamine (0.052 mol) are added to the reaction mixture. After the total 150 min, the temperature is increased to 250° C. over about 15-20 min and is then maintained at the plateau up to the end of the synthesis.

After 2 hours of plateau, the reactor is gradually placed under vacuum over a period of 60 min, and then kept under a partial vacuum in order to limit foaming (36 mBar) for an additional one hour.

At the end of the cycle, the stirring is stopped and the reactor is allowed to cool to room temperature under a nitrogen stream. 192.5 g of polymer are collected. The hyperbranched copolyamide obtained is a whitish solid and will be finely ground for its subsequent use.

### EXAMPLE 11

### 50/50 Water-in-Oil Inverse Emulsion

An aqueous solution comprising 10% by weight of hyperbranched copolyamide of example 10 and 0.6% NaCl is prepared and brought to pH=6.3 with the aid of NaOH.

10 g of this aqueous solution are gradually added to 10 g of the silicone oil Rhodorsil Extrasoft markted by Rhodia. The mixture is sheared with the aid of a paddle frame at 400 revolutions per minute for 1-5 minutes.

Optical microscopy shows that the size of the droplets of this emulsion is less than or equal to 1 μm.

### EXAMPLE 12

### Water-in-Oil-in-Water 45/45/10 Multiple Emulsion

Outer aqueous phase: an aqueous solution comprising 10% by weight of Synperonic PE/F127 marketed by Uniquema and 0.6% of NaCl is prepared.

Emulsification by phase inversion 90/10:

2 g of the outer aqueous phase are added to 18 g of the inverse emulsion of example 11 (inner emulsion) and the whole is sheared with the aid of a paddle frame at 100 revolutions per minute for 2.5 minutes.

### EXAMPLE 13

### 35/65 Water-in-Oil Inverse Emulsion

Inner aqueous phase: an aqueous solution comprising 10% by weight of hyperbranched copolyamide of example 10 and 0.6% of NaCl is prepared and brought to pH=6.3 with the aid of NaOH (or HCl).

7 g of this aqueous solution are gradually added to 13 g of the silicone oil Rhodorsil Extrasoft marketed by Rhodia. The mixture is sheared with the aid of a paddle frame at 400 revolutions per minute for 1-5 minutes.

Optical microscopy shows that the size of the droplets of this emulsion is less than or equal to 1 μm.

### EXAMPLE 14

### Water-in-Oil-in-Water 28/52/20 Multiple Emulsion

Outer aqueous phase: an aqueous solution containing 10% by weight of Synperonic PE/F127 marketed by Uniquema and 0.6% of NaCl is prepared.

Emulsification by phase inversion 80/20:

4 g of the outer aqueous phase are added to 16 g of the inverse emulsion of example 13 and the whole is sheared with the aid of a paddle frame at 100 revolutions per minute for 2.5 minutes.

### EXAMPLE 15

### Multiple Emulsion Comprising a Single Emulsifying Polymer for the Inner Emulsion and the Inverse Emulsion

The procedure is carried out as indicated in examples 11 and 12, the only difference being that the Synperonic PE/F127 is replaced by the hyperbranched copolyamide of example 10.

A stable multiple emulsion is obtained.

### EXAMPLE 16

### Introduction of a Multiple Emulsion into a Detergent Medium

1 g of laundry soap Ariel Regular marketed by Procter & Gamble, Tide marketed by Procter & Gamble is introduced into 100 of water with a TH hardness=30° f., and 0.1 g of the multiple emulsion of example 12 is added as silicone oil equivalent. The medium is stirred with the aid of a magnetic stirrer at 25-30° C. Samples are collected after 2, 20 and 120 minutes, and it is observed under the microscope that the emulsion structure is preserved.

1-17. (canceled)

18. An emulsion comprising an inner phase, an outer phase and an emulsifying polymer, one of the phases being an aqueous phase, wherein the emulsifying polymer is a dendritic polymer.

19. The emulsion as claimed in claim 18, wherein the dendritic polymer is dispersible or soluble in water, at the pH of the emulsion.

20. The emulsion as claimed in claim 18, wherein the dendritic polymer is a hyperbranched polymer comprising hydrophobic groups, and hydrophilic or potentially hydrophilic groups.

21. The emulsion as claimed in claim 18, wherein the emulsion is a direct emulsion, the aqueous phase being the outer phase, and wherein at least some of the hydrophilic or potentially hydrophilic groups are groups present at the polymer chain ends.

22. The emulsion as claimed in claim 18, wherein the emulsion is an inverse emulsion, the aqueous phase being the inner phase, and wherein at least some of the hydrophobic groups are groups present at the polymer chain ends.

23. The emulsion as claimed in claim 18, wherein the emulsion is a multiple emulsion comprising an inner aqueous phase, an intermediate phase and an outer aqueous phase, the inner phase and the intermediate phase constituting an inner inverse emulsion, the intermediate phase and the outer phase constituting an outer direct emulsion, and wherein at least one of the emulsions chosen from the inner inverse emulsion and the outer direct emulsion comprises the dendritic polymer.

24. The emulsion as claimed in claim 23, wherein the outer direct emulsion and the inner inverse emulsion comprise the dendritic polymer.

25. The emulsion as claimed in claim 18, wherein the dendritic polymer is a hyperbranched polyamide or a hyperbranched polyester.

26. The emulsion as claimed in claim 18, wherein the dendritic polymer is a hyperbranched polymer capable of being obtained by a process comprising the following steps:
a) polycondensation, so as to obtain a polymer, of monomers comprising at least one plurifunctional monomer comprising at least three reactive functional groups, of the following formula (I):
A-R—(B)ᵣ (I)
in which formula
f is an integer greater than or equal to 2, preferably ranging from 2 to 10, most particularly equal to 2,
the symbol A represents a reactive functional group or a group carrying a reactive functional group chosen from amino, carboxyl, hydroxyl, oxiranyl, halo and isocyanato functional groups, or precursors thereof,
the symbol B represents a reactive functional group or a group carrying a reactive functional group chosen from amino, carboxyl, hydroxyl, oxiranyl, halo and isocyanato functional groups, or precursors thereof, which is an antagonist of A,
the symbol R represents a linear or branched aliphatic, cycloaliphatic or aromatic polyvalent hydrocarbon residue containing from 1 to 50, optionally interrupted by one or more oxygen, nitrogen, sulfur or phosphorus heteroatoms, said residue optionally carrying functional groups not capable of reacting with the functional groups A and B, and
Step b) optionally at least partial hydrophilic functionalization of the polymer obtained in the polycondensation step.

27. The emulsion as claimed in claim 26, wherein the monomers of step a) comprise:
at least one bifunctional monomer, in linear form, of formula (II) or in the corresponding cyclic form, comprising two polycondensation/polymerization reactive functional groups
A′-R′—B′ (II)
in which formula:
the symbol A′, which is identical to or different from A, represents a reactive functional group chosen from amino, carboxyl, hydroxyl, oxiranyl, halo and isocyanato functional groups, or precursors thereof, which is an antagonist of B and B′,
the symbol B′, which is identical to or different from B, represents a reactive functional group chosen from amino, carboxyl, hydroxyl, oxiranyl, halo and isocyanato functional groups, or precursors thereof, which is an antagonist of A and A′,
the symbol R′, which is identical to or different from R, represents a linear or branched aliphatic, cycloaliphatic or aromatic polyvalent hydrocarbon residue containing from 1 to 50, optionally interrupted by one or more oxygen, nitrogen, sulfur or phosphorus heteroatoms, said residue optionally carrying functional groups not capable of reacting with the functional groups A, A′, B and B′,
the reactive functional group A′ being capable of reacting with the functional group B and/or the functional group B′ by condensation;
the reactive functional group B′ being capable of reacting with the functional group A and/or the functional group A′ by condensation;
and/or at least one “core” monomer of formula (III), comprising at least one functional group capable of reacting, by condensation, with the monomer of formula (I) and/or the monomer of formula (II)
R¹—(B″)ₙ (III)
in which formula
n is an integer greater than or equal to 1, optionally ranging from 1 to 100, most particularly from 1 to 20,
the symbol B″ represents a reactive functional group, which is identical to or different from B or B′, chosen from amino, carboxyl, hydroxyl, oxiranyl, halo and isocyanato functional groups, or precursors thereof, which is an antagonist of A and A′,
the symbol R¹ represents a linear or branched aliphatic, cycloaliphatic or aromatic polyvalent hydrocarbon residue containing from 1 to 50, optionally interrupted by one or more oxygen, nitrogen, sulfur or phosphorus heteroatoms, or an organosiloxane or polyorganosiloxane residue, said residue R¹ optionally carrying functional groups not capable of reacting with the functional groups A, A′, B, B′ and B″,
the reactive functional group B″ being capable of reacting with the functional group A and/or the functional group A′ by condensation;
and/or at least one “chain limiting” mono-functional monomer of formula (IV)
A″-R² (IV)
in which formula
the symbol A″ represents a reactive functional group, which is identical to or different from A or A′, chosen from amino, carboxyl, hydroxyl, oxiranyl, halo and isocyanato functional groups, or precursors thereof, which is an antagonist of B, B′ and B″,
the symbol R² represents a linear or branched aliphatic, cycloaliphatic or aromatic polyvalent hydrocarbon residue containing from 1 to 50, optionally interrupted by one or more oxygen, nitrogen, sulfur or phosphorus heteroatoms, or an organosiloxane or polyorganosiloxane residue, said residue R² optionally carrying functional groups not capable of reacting with the functional groups A, A′, A″, B, B′ and B″,
the reactive functional group A″ being capable of reacting with the functional group B and/or the functional group B′ and/or the functional group B″ by condensation; and
at least one of the reactive functional groups of at least one of the monomers of formula (II), (III) or (IV) being capable of reacting with a functional group which is an antagonist of the plurifunctional monomer of formula (I).

28. The emulsion as claimed in claim 26, wherein the functional groups A, A′ A″, and B, B′, B″ are the reactive functional groups or the groups carrying reactive functional groups which are amino, carboxyl, hydroxyl, oxiranyl functional groups, or precursors thereof.

29. The emulsion as claimed in claim 26, wherein at least one chain limiting monomer is further used, said monomer being hydrophilic or potentially hydrophilic.

30. The emulsion as claimed in claim 18, being a formulation of a cosmetic product, of a detergent product, of a paint or of a coating.

31. The emulsion as claimed in claim 30, wherein the cosmetic product formulation is a skin or hair care product.

32. An emulsifying agent comprising a dendritic polymer as defined in claim 18.

33. A formulation of a cosmetic product, of a detergent product, of a paint or of a coating, comprising a dendritic polymer as defined in claim 18.

34. The formulation as claimed in claim 33, wherein the cosmetic product formulation is a skin or hair care product.
